(19) United States
(12) Patent Application Publication
(10) Pub. No.: US 2026/0240610 A1
(43) Pub. Date: Aug. 20, 2026

(54) COMPUTED TOMOGRAPHY (CT) SYSTEM FOR GENERATING A CT IMAGE OF A REGION OF INTEREST OF A SUBJECT AND AN INTERVENTIONAL DEVICE USING SPARSE-VIEW CT PROJECTION DATA DURING A CT-GUIDED INTERVENTIONAL PROCEDURE

(71) Applicants: GE Precision Healthcare LLC, Waukesha, WI (US); Emory University, Atlanta, GA (US)

(72) Inventors: Amir Pourmorteza, Atlanta, GA (US); Bruno De Man, Clifton Park, NY (US); Eri Haneda, Niskayuna, NY (US); Cynthia Davis, Niskayuna, NY (US); Biju Jacob, Niskayuna, NY (US); Pengwei Wu, Clifton Park, NY (US); Sathish Ramani, Niskayuna, NY (US); Jiayong Zhang, Niskayuna, NY (US); Jed Pack, Glenville, NY (US); Grigorios Karageorgos, Niskayuna, NY (US); Stephen Araujo, Niskayuna, NY (US)

(21) Appl. No.: 19/054,289

(22) Filed: Feb. 14, 2025

(30) Foreign Application Priority Data
Feb. 14, 2025 (GR) 20250100129

(51) Int. Cl. A61B 34/30; A61B 6/00; A61B 6/03; A61B 6/46; A61B 34/20; G06T 11/00

(52) U.S. Cl. CPC A61B 34/30; A61B 6/032; A61B 6/461; A61B 6/54; G06T 12/20; A61B 2034/2065; G06T 2210/41; G06T 2211/40

(57) ABSTRACT

A computed tomography (CT) system may acquire sparse-view CT projection data of a region of interest of a subject and an interventional device provided within the region of interest of the subject during a CT-guided interventional procedure by pulsing an X-ray source. The CT system may generate one or more time-resolved CT images of the region of interest and the interventional device using the sparse-view CT projection data and a reconstruction technique. The CT system may utilize the CT image of the region of interest and the interventional device during the CT-guided interventional procedure, such as by displaying the CT image via a display of the CT system or by providing the CT image to a robotic surgical system that is configured to perform the CT-guided interventional procedure.

### TECHNICAL FIELD

The present disclosure relates to a CT system for generating a CT image of one or more regions of interest of a subject and one or more interventional devices using sparse-view CT projection data during a CT-guided interventional procedure.

### BACKGROUND

A CT system may include a gantry and a rotational frame that support an X-ray source and an X-ray detector. The rotational frame may rotate the X-ray source and the X-ray detector around a subject that is positioned on a table. The X-ray source may emit X-ray radiation in the form of an X-ray radiation beam towards the subject and the X-ray detector. The X-ray detector may detect X-ray radiation that is emitted by the X-ray source and that is attenuated by the subject. CT projection data from the detector at a particular gantry angle may be referred to as a “view.” A “scan” of the subject may include a set of views made at different gantry angles, or “view angles,” during one or more rotations of the X-ray source and the X-ray detector. For full-view imaging, the CT system may acquire a relatively large number of views, such as one thousand views, nine hundred views, or the like.

An interventional procedure (e.g., catheterization, needle biopsy, robotic surgery, or the like) may generally involve the navigation of one or more interventional devices (e.g., a catheter, a needle, or the like) through one or more regions of interest of a subject. To assist with the navigation of the interventional device, a medical imaging system may acquire real-time medical images that depict the region of interest and the interventional device. For example, an X-ray system may acquire two-dimensional (2D) X-ray images of the region of interest and the interventional device that depict the position of the interventional device in relation to the relevant anatomy of the region of interest. A CT system may acquire relatively more comprehensive three-dimensional (3D) images of the region of interest and the interventional device as compared to 2D X-ray images. However, utilization of CT systems during interventional procedures may be prohibitive due to the higher radiation doses associated with CT data acquisition for full-view imaging as compared to 2D X-ray fluoroscopy. Further, the time required for full-view CT data acquisition and CT image generation may be prohibitive during interventional procedures.

### SUMMARY

This summary introduces concepts that are described in more detail in the detailed description. It should not be used to identify essential features of the claimed subject matter, nor to limit the scope of the claimed subject matter.

In an aspect, a computed tomography (CT) system may include a memory configured to store instructions; and one or more processors configured to execute the instructions to: acquire sparse-view CT projection data of a region of interest of a subject and an interventional device provided within the region of interest of the subject during a CT-guided interventional procedure by pulsing an X-ray source; generate a CT image of the region of interest and the interventional device using the sparse-view CT projection data and a reconstruction technique; and utilize the CT image of the region of interest and the interventional device during the CT-guided interventional procedure.

In another aspect, a method may include acquiring sparse-view computed tomography (CT) projection data of a region of interest of a subject and an interventional device provided within the region of interest of the subject during a CT-guided interventional procedure by pulsing an X-ray source; generating a CT image of the region of interest and the interventional device using the sparse-view CT projection data and a reconstruction technique; and utilizing the CT image of the region of interest and the interventional device during the CT-guided interventional procedure.

In yet another aspect, a non-transitory computer-readable medium may store instructions that, when executed by one or more processors, cause the one or more processors to: acquire sparse-view computed tomography (CT) projection data of a region of interest of a subject and an interventional device provided within the region of interest of the subject during a CT-guided interventional procedure by pulsing an X-ray source; generate a CT image of the region of interest and the interventional device using the sparse-view CT projection data and a reconstruction technique; and utilize the CT image of the region of interest and the interventional device during the CT-guided interventional procedure.

### BRIEF DESCRIPTION OF DRAWINGS

FIG. 1 is a diagram of an example system for utilizing a CT image of a region of interest of a subject and an interventional device that is generated using sparse-view CT projection data during a CT-guided interventional procedure.

FIG. 2 is a perspective view of an example CT system for generating a CT image of a region of interest of a subject and an interventional device using sparse-view CT projection data during a CT-guided interventional procedure.

FIG. 3 is a diagram of components of an example CT system for generating a CT image of a region of interest of a subject and an interventional device using sparse-view CT projection data during a CT-guided interventional procedure.

FIG. 4 is a diagram of components of an example robotic surgical system for utilizing a CT image of a region of interest of a subject and an interventional device that is generated using sparse-view CT projection data during a CT-guided interventional procedure.

FIG. 5 is a flowchart of an example process for generating a CT image of a region of interest of a subject and an interventional device using sparse-view CT projection data during a CT-guided interventional procedure.

FIGS. 6A and 6B are diagram of example tube currents of an x-ray source of a CT system for particular gantry angles.

FIGS. 7A and 7B are diagrams of an example process for generating a CT image using the sparse-view CT projection data, a full-view CT image, and a reconstruction of difference technique.

FIG. 8 is a diagram of an example process for generating a CT image of the region of interest and the interventional device using the sparse-view CT projection data, a full-view CT image, and a reconstruction technique.

FIG. 9 is a diagram of an example process for generating a CT image of the region of interest and the interventional device using the sparse-view CT projection data, a full-view CT image, and a weighted iterative reconstruction technique.

FIG. 10 is a diagram of an example process for generating a CT image of the region of interest and the interventional device using the sparse-view CT projection data and a stacked back projection technique.

FIG. 11 is a diagram of an example process for generating a CT image of the region of interest and the interventional device using the sparse-view CT projection data and an AI technique.

FIG. 12 is a diagram of an example process for generating a CT image of a region of interest of a subject and an interventional device using sparse-view CT projection data during a CT-guided interventional procedure.

FIG. 13 is a diagram of an example process for training, deploying, and monitoring an AI model for generating a CT image of a region of interest of a subject and an interventional device using sparse-view CT projection data during a CT-guided interventional procedure.

### DETAILED DESCRIPTION

As addressed above, a CT system may acquire relatively more comprehensive and higher quality 3D images of a region of interest of a subject as compared to 2D X-ray images. However, utilization of CT systems during interventional procedures may be prohibitive due to the higher radiation doses associated with CT data acquisition as compared to 2D X-ray fluoroscopy, and due to the relatively longer time required to acquire full-view CT projection data.

Some embodiments herein provide a CT system that may acquire sparse-view CT projection data of a region of interest of a subject and an interventional device provided within the region of interest of the subject during a CT-guided interventional procedure by pulsing an X-ray source, generate a CT image of the region of interest and the interventional device using the sparse-view CT projection data and a reconstruction technique, and utilize the CT image of the region of interest and the interventional device during the CT-guided interventional procedure. In this way, some embodiments herein permit the implementation of a CT system for CT-guided interventional procedures by utilizing the acquisition of sparse-view CT projection data and the reconstruction of real-time CT images during the CT-guided interventional procedures. By utilizing sparse-view CT projection data, embodiments herein improve the safety of the subject and clinicians by reducing radiation exposure as compared to situations in which full-view CT projection data is acquired.

The embodiments herein provide an improvement to the technical field of CT imaging and an improvement to interventional procedures by providing the real-time 3D visualization of an interventional device in a region of interest of a subject using sparse-view CT projection data. Further, the embodiments herein provide an improvement to CT systems by permitting the generation of a CT image of a region of interest and an interventional device using the sparse-view CT projection data and a reconstruction technique, and by permitting the utilization of the CT image of the region of interest and the interventional device during a CT-guided interventional procedure. Further, the embodiments herein may reduce the time to reach a region of interest using an interventional device because it might be easier for a clinician or a robotic surgical system to navigate the interventional device to the region of interest with the utilization of 3D CT images during the CT-guided interventional procedure. Further still, the embodiments herein may enable more complex interventional procedures that would have been impractical, or otherwise impossible, with only 2D X-ray fluoroscopy information. Further still, the embodiments herein may reduce the total amount of contrast agent that needs to be injected during interventional procedures. Further still, the embodiments herein may enable automated interventional procedures that utilize automated robotic surgical systems. Further still, some embodiments herein may provide increased access to interventional procedures because of the large installed base of CT systems as compared to other medical imaging systems.

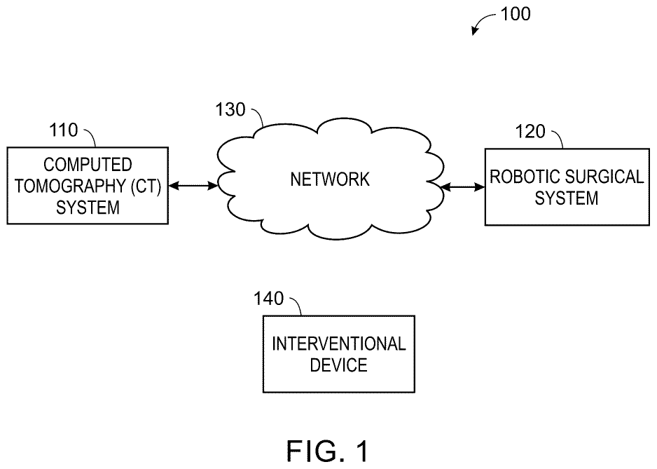

FIG. 1 is a diagram of an example system 100 for utilizing a CT image of a region of interest of a subject and an interventional device that is generated using sparse-view CT projection data during a CT-guided interventional procedure. As shown in FIG. 1, the system 100 may include a CT system 110, a robotic surgical system 120, a network 130, and an interventional device 140.

The CT system 110 may be configured to acquire sparse-view CT projection data of a region of interest of a subject and the interventional device 140 provided within the region of interest of the subject during a CT-guided interventional procedure, generate a CT image of the region of interest and the interventional device 140 using the sparse-view CT projection data and a reconstruction technique, and utilize the CT image of the region of interest and the interventional device 140 during the CT-guided interventional procedure. For example, the CT system 110 may be a photon-counting CT system, a dual-energy CT system, sequential CT system, a spiral CT system, a stationary CT system, or the like. Although the embodiments herein describe the utilization of a particular CT system 110, the embodiments herein may be applicable to other types of medical imaging systems, such as X-ray imaging systems including C-Arm systems, O-ring systems, cone beam CT systems, or the like. Further, although the embodiments herein describe the utilization of sparse-view CT projection data of a region of interest of a subject and the interventional device 140 provided within the region of interest of the subject during a CT-guided interventional procedure, it should be understood that the embodiments herein are applicable to situations, or procedures, that do not involve the navigation of the interventional device 140 through a region of interest.

The robotic surgical system 120 may be configured to perform an interventional procedure using the interventional device 140. For example, the robotic surgical system 120 may be an autonomous system, a manual system that is controlled by a clinician, a semi-autonomous system, or the like.

The network 130 may permit communication between the CT system 110 and the robotic surgical system 120. For example, the network 130 may be a local area network (LAN), a wide area network (WAN), a metropolitan area network (MAN), a cellular network, a private network, an ad hoc network, an intranet, the Internet, a fiber optic-based network, a wired network, a wireless network, or the like, and/or a combination of these or other types of networks.

The interventional device 140 may be any device that can be navigated through a region of interest of a subject. For example, the interventional device 140 may be a catheter, a needle, a guidewire, a trocar, a cannula, or the like. The interventional device 140 may be used for various interventional procedures involving the region of interest. For example, a catheter may be used for delivering a stent to an occluded blood vessel, inserting a mitral valve clip, closing a left atrial appendage, ablating tissue, analyzing cardiac function, removing a thrombus from an occluded blood vessel, or the like. Alternatively, the interventional device 140 may be an implantable device that is to be implanted in the subject. For example, the interventional device 140 may be a pacemaker, a stent, a defibrillator, a left ventricular assist device, a valve clip, or the like. Alternatively, the interventional device 140 may be any object that can be navigated throughout the region of interest of the subject.

The subject may be a patient, an animal, a phantom, an object, or the like. The region of interest of the subject may be any anatomical region, or regions, of the subject. For example, the region of interest may be heart, the lungs, the brain, the liver, the pancreas, or the like. The CT-guided interventional procedure may be any type of interventional procedure that utilizes CT images to guide the interventional device 140 to a target area in a region of interest of a subject. For example, the CT-guided interventional procedure may be a biopsy in which a tissue sample is extracted using a needle, an ablation procedure in which an ablation device is used to ablate tissue, a drainage procedure in which a catheter is used to drain fluid, an incision procedure in which is scalpel is used to make an incision, or the like.

The number and arrangement of the system 100 are provided as an example. In practice, the system 100 may include additional systems, fewer systems, different systems, or differently arranged systems than those shown in FIG. 1. Additionally, or alternatively, a set of systems (e.g., one or more systems) of the system 100 may be integrated into a single system, and/or perform one or more functions described as being performed by another system, or set of systems, of the system 100.

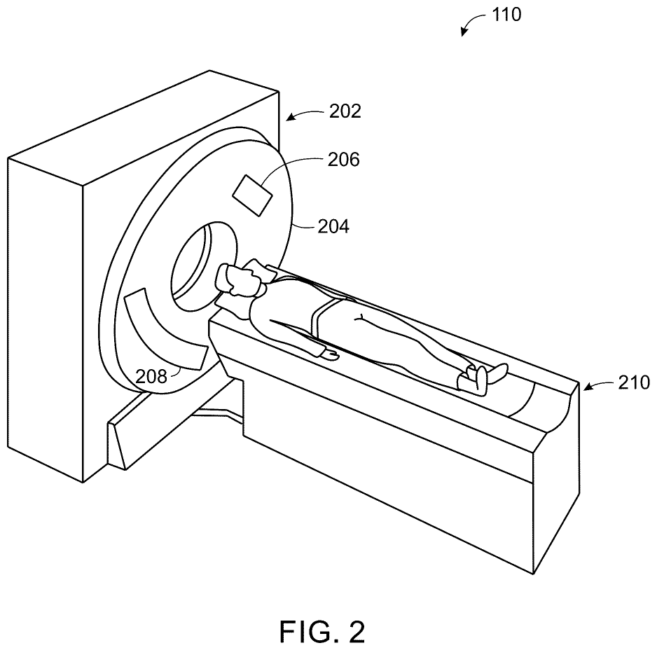

FIG. 2 is a perspective view of an example CT system 110 for generating a CT image of a region of interest of a subject and an interventional device 140 using sparse-view CT projection data during a CT-guided interventional procedure. As shown in FIG. 2, the CT system 110 may include a gantry 202, a rotational frame 204, an X-ray source 206, an X-ray detector 208, and a table 210.

The gantry 202 may be configured to support the rotational frame 204, the X-ray source 206, and the X-ray detector 208. The rotational frame 204 may be configured to rotate the X-ray source 206 and the X-ray detector 208 around a subject that is positioned on the table 210. The X-ray source 206 may be configured to emit X-ray radiation in the form of an X-ray radiation beam towards the subject and the X-ray detector 208. According to an embodiment, the X-ray source 206 may include one or more X-ray tubes that are configured to generate X-ray radiation. An X-ray tube may include a grid electrode positioned adjacent to a cathode that permits the X-ray radiation beam to be selectively modulated. The X-ray detector 208 may be configured to detect X-ray radiation emitted by the X-ray source 206 and attenuated by the subject. According to an embodiment, the CT system 110 may include one or more X-ray sources 206 and/or one or more X-ray detectors 208 that are configured to acquire CT projection data at the same or different energy levels. According to an embodiment, the X-ray source 206 may be configured for dual-energy spectral imaging by rapid peak kilovoltage (kVp) switching. Additionally, or alternatively, the X-ray detector 208 may be a photon counting detectors, a dual-layer detector, or a multi-layer detector that is configured to differentiate X-ray photons having different energies. The table 210 may be configured to support the subject during a scan of the subject. During a scan of the subject, the gantry 202 may rotate the rotational frame 204 around the subject to change an angle at which an X-ray radiation beam emitted by the X-ray source 206 intersects the subject. The X-ray detector 208 may acquire CT projection data by detecting radiation of the X-ray radiation beam. In another embodiment, the CT system 110 may be a stationary (non-rotating) CT system 110 in which an array of X-ray sources 206 allows emitted X-rays from different view angles without mechanical rotation.

The CT system 110 may be configured to control the X-ray source 206 to pulse the X-ray source 206. For example, the CT system 110 may control the X-ray source 206 to pulse the X-ray source 206 on and off to acquire sparse-view CT projection data. For instance, the CT system 110 may pulse the X-ray source 206 by switching the X-ray source 206 on such that the X-ray source 206 generates an X-ray radiation beam, and switching the X-ray source 206 off such that the X-ray source 206 does not generate an X-ray radiation beam. Alternatively, the CT system 110 may control the X-ray source 206 to pulse the X-ray source 206 such that the X-ray source 206 generates an X-ray radiation beam having different photon flux levels to acquire sparse-view CT projection data. The CT system 110 may pulse the X-ray source 206 in a rapid manner. That is, the CT system 110 may pulse the X-ray source 206 such that the X-ray source 206 is generating an X-ray radiation beam, or is generating an X-ray radiation beam having a relatively high photon flux level, for a threshold amount of time for each view. For instance, the threshold amount of time may be one hundred microseconds, two hundred microseconds, three hundred microseconds, five hundred microseconds, etc.

According to an embodiment, the X-ray source 206 may project a cone-shaped X-ray radiation beam which is defined with respect to an X-Y-Z Cartesian coordinate system and generally referred to as an “imaging volume.” The X-ray radiation beam may pass through the subject that is being imaged, and may be attenuated by the subject. After being attenuated by the subject, the X-ray radiation beam impinges on an array of detector elements of the X-ray detector 208. The intensity of the attenuated X-ray radiation beam received at the detector array of the X-ray detector 208 may depend on the amount of attenuation of the X-ray radiation beam by the object. Each detector element of the array of the X-ray detector 208 may generate a separate electrical signal that is a measurement of the X-ray radiation beam attenuation at the location of the respective detector element of the X-ray detector 208. The attenuation measurements from the detector elements may be acquired separately to generate a transmission profile.

According to an embodiment, the X-ray source 206 and the X-ray detector 208 are rotated with the gantry 202 within the imaging volume and around the subject to be imaged such that an angle at which the X-ray radiation beam intersects the subject changes. CT projection data from the detector array of the X-ray detector 208 at a particular gantry angle may be referred to as a “view.” A “scan” of the subject includes a set of views made at different gantry angles, or “view angles,” during one or more rotations of the X-ray source 206 and the X-ray detector 208.

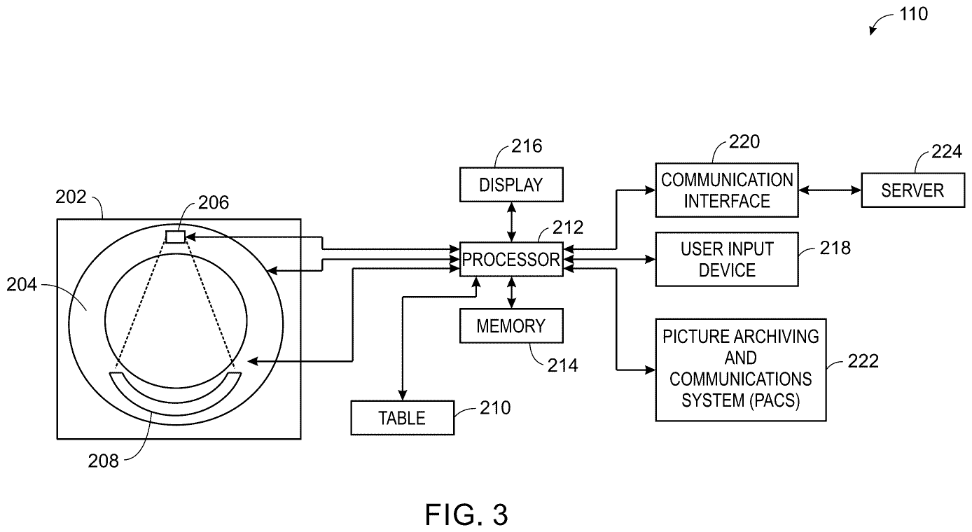

FIG. 3 is a diagram of components of an example CT system 110 for generating a CT image of a region of interest of a subject and an interventional device using sparse-view CT projection data during a CT-guided interventional procedure. As shown in FIG. 3, the CT system 110 may include a gantry 202, a rotational frame 204, an X-ray source 206, an X-ray detector 208, a table 210, a processor 212, a memory 214, a display 216, a user input device 218, a communication interface 220, a picture archiving and communications system (PACS) 222, and a server 224.

The processor 212 may be configured to control operations of the CT system 110. For example, the processor 212 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or the like. The processor 212 may be implemented in hardware, firmware, or a combination of hardware and software. The processor 212 may include one or more processors 212 configured to perform the operations described herein. For example, a single processor 212 may be configured to perform all of the operations described herein. Alternatively, multiple processors 212, collectively, may be configured to perform all of the operations described herein, and each of the multiple processors 212 may be configured to perform a subset of the operations described herein. For example, a first processor 212 may perform a first subset of the operations described herein, a second processor 212 may be configured to perform a second subset of the operations described herein, etc.

The processor 212 may be configured to control the gantry 202, movement of the rotational frame 204, the X-ray source 206, the X-ray detector 208, and movement of the table 210. The processor 212 may provide signals to the X-ray source 206 to cause the X-ray source 206 to emit X-ray radiation in the form of an X-ray radiation beam towards the subject and the X-ray detector 208 during a scan, and may receive CT projection data generated during the scan. The processor 212 may generate a sinogram using the CT projection data. The sinogram may be a one-dimensional (1D) sinogram, a 2D sinogram, a 3D sinogram, or the like. The processor 212 may generate a CT image based on the sinogram. For example, the processor 212 may generate the CT image using a reconstruction technique, such as filtered back projection (FBP), advanced statistical iterative reconstruction (ASIR), conjugate gradient (CG), maximum likelihood expectation maximization (MLEM), model-based iterative reconstruction (MBIR), or the like.

The processor 212 may control the gantry 202, movement of the rotational frame 204, and/or the table 210 to perform a scan of a particular region of interest of the subject. According to an embodiment, the processor 212 may determine a position of the interventional device 140 in the region of interest of the subject, and control the gantry 202, movement of the rotational frame 204, and/or the table 210 to position the X-ray source 206 relative to the interventional device 140.

The memory 214 may be configured to store information and/or instructions for use by the processor 212. The memory 214 may be a non-transitory computer-readable medium. For example, the memory 214 may be a random access memory (RAM), a read only memory (ROM), a flash memory, a magnetic memory, an optical memory, or the like. The memory 214 may be configured to store instructions that, when executed by the processor 212, cause the processor 212 to perform the operations described herein.

The display 216 may be configured to display information. For example, the display 216 may be a monitor, a light-emitting diode (LED) display, a cathode ray tube, a projector display, a touchscreen, tablet computer, mobile phone, or the like. The display 216 may display CT images in substantially real-time. For example, the display 216 may display the CT images within one second, two seconds, five seconds, etc., of the CT images being generated, a scan or rotation being completed, CT projection data being acquired, or the like.

The user input device 218 may be configured to receive a user input, and provide the user input to the processor 212. For example, the user input device 218 may be a user interface, a touch screen display, a keyboard, a keypad, a mouse, a button, a switch, a microphone, or the like. Additionally, or alternatively, the user input device 218 may be configured to sense information. For example, the user input device 218 may sense information from an electro-magnetic positioning system, an inertial measurement system, an accelerometer, a gyroscope, an actuator, or the like.

The communication interface 220 may be configured to enable the processor 212 to communicate with other devices, such as via a wired connection, a wireless connection, or a combination of wired and wireless connections. For example, the communication interface 220 may include an Ethernet interface, an optical interface, a coaxial interface, an infrared interface, a radio frequency (RF) interface, a universal serial bus (USB) interface, a wireless fidelity (Wi-Fi) interface, a cellular network interface, or the like. The PACS 222 may be configured to communicate with external systems and/or networks to permit users at various locations to access the CT image. The server 224 may be configured to store one or more AI models as described herein. For example, the server 224 may be an on-premises server, a cloud server, a virtual machine, or the like.

The number and arrangement of the components of the CT system 110 are provided as an example. In practice, the CT system 110 may include additional components, fewer components, different components, or differently arranged components than those shown in FIG. 3. Additionally, or alternatively, a set of components (e.g., one or more components) of the CT system 110 may be integrated into a single component, and/or perform one or more functions described as being performed by another components, or set of components, of the CT system 110.

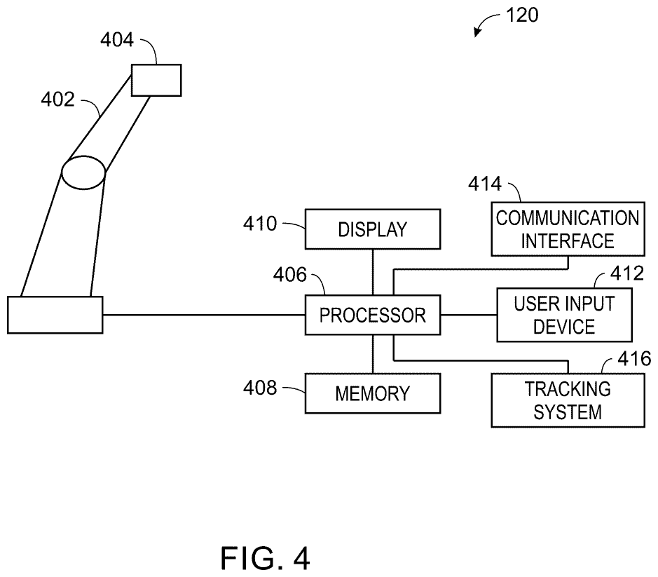

FIG. 4 is a diagram of components of an example robotic surgical system 120 for utilizing a CT image of a region of interest of a subject and an interventional device 140 that is generated using sparse-view CT projection data during a CT-guided interventional procedure. As shown in FIG. 4, the robotic surgical system 120 may include an arm 402, an end effector 404, a processor 406, a memory 408, a display 410, a user input device 412, a communication interface 414, and a tracking system 416.

The arm 402 may be configured to support the end effector 404 and manipulate the end effector 404. For example, the arm 402 may include any number of linkages and may include any number of degrees of freedom. The end effector 404 may be a medical instrument, a tool, a grip, or the like. Additionally, or alternatively, the end effector 404 may be, or might include, the interventional device 140. The robotic surgical system 120 may include any number of arms 402 and/or end effectors 404.

The processor 406 may be configured to control the robotic surgical system 120. For example, the processor 406 may be a CPU, a GPU, an APU, a microprocessor, a microcontroller, a DSP, an FPGA, an ASIC, or the like. The processor 406 may be implemented in hardware, firmware, or a combination of hardware and software. The processor 406 may include one or more processors 406 configured to perform the operations described herein. For example, a single processor 406 may be configured to perform all of the operations described herein. Alternatively, multiple processors 406, collectively, may be configured to perform all of the operations described herein, and each of the multiple processors 406 may be configured to perform a subset of the operations described herein. For example, a first processor 406 may perform a first subset of the operations described herein, a second processor 406 may be configured to perform a second subset of the operations described herein, etc.

The processor 406 may be configured to perform the CT-guided interventional procedure autonomously, such as without any input from a clinician. Alternatively, the processor 406 may be configured to perform the CT-guided interventional procedure based on one or more inputs received from a clinician via the user input device 412.

According to an embodiment, the processor 406 may receive a CT image from the CT system 110 based on the CT system 110 generating the CT image. The processor 406 may be configured to perform the CT-guided interventional procedure using the CT image received from the CT system 110.

The memory 408 may be configured to store information and/or instructions for use by the processor 406. The memory 408 may be a non-transitory computer-readable medium. For example, the memory 408 may be a RAM, a ROM, a flash memory, a magnetic memory, an optical memory, or the like. The memory 408 may be configured to store instructions that, when executed by the processor 406, cause the processor 406 to perform the operations described herein.

The display 410 may be configured to display information. For example, the display 410 may be a monitor, an LED display, a cathode ray tube, a projector display, a touchscreen, tablet computer, mobile phone, or the like.

The user input device 412 may be configured to receive a user input, and provide the user input to the processor 406. For example, the user input device 412 may be a user interface, a touch screen display, a keyboard, a keypad, a mouse, a button, a switch, a microphone, or the like. Additionally, or alternatively, the user input device 412 may be configured to sense information. For example, the user input device 412 may sense information from an electro-magnetic positioning system, an inertial measurement system, an accelerometer, a gyroscope, an actuator, or the like.

The communication interface 414 may be configured to enable the processor 406 to communicate with other systems, such as via a wired connection, a wireless connection, or a combination of wired and wireless connections. For example, the communication interface 414 may include an Ethernet interface, an optical interface, a coaxial interface, an infrared interface, an RF interface, a USB interface, a Wi-Fi interface, a cellular network interface, or the like.

The tracking system 416 may be configured to track the end effector 404 and/or the interventional device 140. For example, the tracking system 416 may be an electromagnetic tracking system, an optical tracking system, an acoustic tracking system, an inertial tracking system, an ultrasound tracking system, or the like.

The number and arrangement of the components of the robotic surgical system 120 are provided as an example. In practice, the robotic surgical system 120 may include additional components, fewer components, different components, or differently arranged components than those shown in FIG. 4. Additionally, or alternatively, a set of components (e.g., one or more components) of the robotic surgical system 120 may be integrated into a single component, and/or perform one or more functions described as being performed by another components, or set of components, of the robotic surgical system 120.

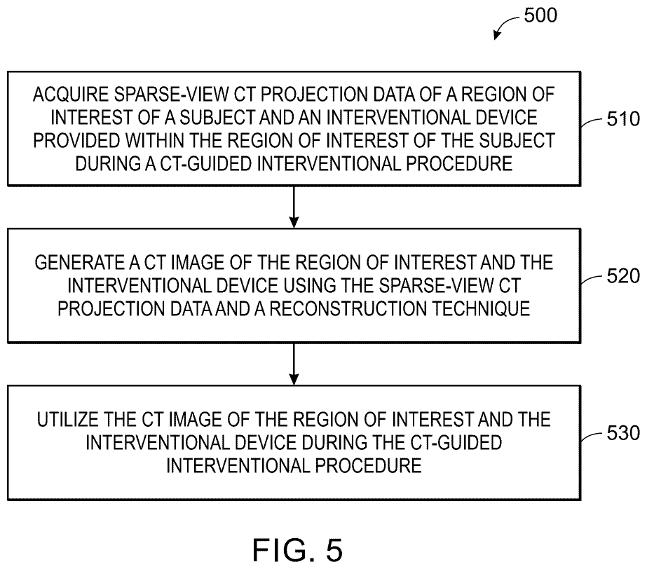

FIG. 5 is a flowchart of an example process 500 for generating a CT image of a region of interest of a subject and an interventional device 140 using sparse-view CT projection data during a CT-guided interventional procedure.

As shown in FIG. 5, the process 500 may include acquiring sparse-view CT projection data of a region of interest of a subject and an interventional device 140 provided within the region of interest of the subject during a CT-guided interventional procedure (operation 510). For example, the CT system 110 may be configured to acquire sparse-view CT projection data of the region of interest of the subject and the interventional device 140 provided within the region of interest of the subject.

As used herein, “sparse-view CT projection data” may refer to CT projection data that is acquired for less than an entire set of gantry angles that the CT system 110 is configured to acquire CT projection data, or may refer to CT projection data that is acquired for less than a threshold number of gantry angles, or view angles, per rotation. For example, sparse-view CT projection data may be acquired for fifty gantry angles, ten gantry angles, three gantry angles, two gantry angles, or the like, per rotation. As used herein “full-view CT projection data” may refer to CT projection data that is acquired for an entire set of gantry angles that the CT system 110 is configured to acquire CT projection data, or may refer to CT projection data that is acquired for greater than a threshold number of gantry angles, or view angles, per rotation. For example, full-view CT projection data may be acquired for two thousand gantry angles, one thousand gantry angles, nine hundred gantry angles, eight hundred gantry angles, or the like, per rotation. According to an embodiment, sparse-view CT projection data may satisfy a predetermined relationship as compared to full-view CT projection data. For example, sparse-view CT projection data may be less than half of the full-view CT projection data, less than five percent of the full-view CT projection data, less than two percent of the full-view CT projection data, less than one percent of the full-view CT projection data, or the like, per rotation.

According to an embodiment, the CT system 110 may be configured to acquire the sparse-view CT projection data based on a predetermined timeframe. For example, the CT system 110 may be configured to acquire the sparse-view CT projection data every minute, every thirty seconds, every ten seconds, every second, or the like. Additionally, or alternatively, the CT system 110 may be configured to acquire the sparse-view CT projection data based on an event. For example, the CT system 110 may be configured to acquire the sparse-view CT projection data based on an initialization of the CT-guided interventional procedure, based on the introduction of the interventional device 140 in the region of interest, based on motion of the interventional device 140 exceeding a threshold, or the like. Additionally, or alternatively, the CT system 110 may be configured to acquire the sparse-view CT projection data based on an input received via the user input device 218. For example, a clinician may interact with the user input device 218 to cause the CT system 110 to acquire the sparse-view CT projection data.

According to an embodiment, the CT system 110 may be configured to acquire the sparse-view CT projection data by acquiring a predetermined number of views per rotation. For example, the CT system 110 may acquire two hundred views per rotation, one hundred views per rotation, fifty views per rotation, ten views per rotation, three views per rotation, two views per rotation, or the like. According to an embodiment, the CT system 110 may be configured to acquire the sparse-view CT projection data by acquiring views at a predetermined angular interval of gantry angles. For example, the CT system 110 may acquire CT projection at an angular interval of 1°, 2°, 3°, 5°, 50°, 137°, etc.

According to an embodiment, the CT system 110 may be configured to acquire the sparse-view CT projection data using an angular sequence. The angular sequence may define the gantry angles for which sparse-view CT projection data is to be acquired by the CT system 110. According to an embodiment, the angular sequence may be determined based on a binary sequence in which digits of a binary number are incremented in reverse. That is, the digits of the binary number may be incremented from left to right instead of right to left. For example, a first binary number of “0000” may correspond to a decimal number of “0” which in turn corresponds to a gantry angle of “0°,” a second binary number of “1000” may be generated by incrementing the first binary number in reverse and may correspond to a decimal value of “8” which in turn corresponds to a gantry angle of “8°,” a third binary number of “0100” may be generated by incrementing the second binary number in reverse and may correspond to a decimal value of “4” which in turn corresponds to gantry angle of “4°,” a fourth binary number of “1100” may be generated by incrementing the third binary number in reverse and may correspond to a decimal value of “12” which in turn corresponds to a gantry angle of “12°,” a fifth binary number of “0010” may be generated by incrementing the fourth binary number in reverse and may correspond to a decimal value of “2” which in turn may correspond to a gantry angle of “2°,” etc. Additionally, or alternatively, the angular sequence may be determined based on gantry angles and an angular interval that is based on the golden ratio (0.5+sqrt(5)/2), a silver ratio, or similar irrational number based phyllotaxic arrangements. For example, each successive gantry angle may be increased by 360° divided by the golden ratio. In this way, a first gantry angle may be 0°, a second gantry angle may be ~138°, a third gantry angle may be ~275°, a fourth gantry angle may be ~53°, a fifth gantry angle may be ~190°, a sixth gantry angle may be ~328°, etc. Additionally, or alternatively, the angular sequence may be determined based on a time-sequential sampling sequence, an ordered subsets sampling sequence, an MRI-inspired golden-angle radial sparse parallel (GRASP) sequence, or the like. Additionally, or alternatively, the angular sequence may be determined using a set of uniformly spaced gantry angles for a full-scan view range (e.g., 360°). For example, a first gantry angle may be 0°, a second gantry angle may be 3.6°, a third gantry angle may be 7.2°, a fourth gantry angle may be 10.8°, etc. Restated, the CT system 110 may divide 360° by a particular number of views to determine the angular interval (e.g., 360°÷100 views=3.6°). Additionally, or alternatively, the angular sequence may be determined using a set of uniformly spaced gantry angles for a half-scan view range (e.g., 180°). For example, a first gantry angle may be 0°, a second gantry angle may be 2.3°, a third gantry angle may be 4.6°, etc. Restated, the CT system 110 may divide the combination of 180° and a fan angle (e.g., 50°) by a particular number of views to determine the angular interval (e.g., (180°+50°)÷100 views=2.3°). Additionally, or alternatively, the angular sequence may be determined using a set of uniformly spaced gantry angles for a limited view range (e.g., 90°). For example, a first gantry angle may be 0°, a second gantry angle may be 1.8°, a third gantry angle may be 3.6°, etc. Restated, the CT system 110 may divide 90° by a particular number of views to determine the angular interval (e.g., 90°÷50 views=1.8°).

According to an embodiment, the CT system 110 may be configured to acquire the sparse-view CT projection data by pulsing the X-ray source 206. For example, the CT system 110 may pulse the X-ray source 206 by modulating intensity, energy, and/or photon flux of an X-ray radiation beam generated by the X-ray source 206 according to the angular sequence. As a particular example, the CT system 110 may pulse the X-ray radiation beam by adjusting a tube current of an X-ray tube of the X-ray source 206. The CT system 110 may be configured to control the X-ray source 206 to pulse the X-ray source 206 to acquire the sparse-view CT projection data. For example, the CT system 110 may control the X-ray source 206 to pulse the X-ray source 206 on and off in a rapid manner. Alternatively, the CT system 110 may control the X-ray source 206 to pulse the X-ray source 206 between generating X-ray radiation beams of a low photon flux level and a high photon flux level. According to an embodiment, the CT system 110 may modulate the X-ray radiation beam such that the X-ray radiation beam is only generated for gantry angles corresponding to the angular sequence. As an example, if the angular sequence is 0°, 138°, 275°, etc., then the CT system 110 may generate an X-ray radiation beam at 0°, might not generate an X-ray radiation beam between 1°and 137°, may generate an X-ray radiation beam at 138°, etc. Alternatively, the CT system 110 may modulate the X-ray radiation beam such that the X-ray radiation beam is generated at a first photon flux level for gantry angles corresponding to the angular sequence and is generated at a second photon flux level, that is lower than the first photon flux level, for gantry angles that do not correspond to the angular sequence. For example, if the angular sequence is 0°, 138°, 275°, etc., then the CT system 110 may generate an X-ray radiation beam having a first photon flux level at 0°, may generate an X-ray radiation beam having a second photon flux level at one or more gantry angles between 1° and 137°, may generate an X-ray radiation beam having the first photon flux level at 138°, etc. In this case, the second photon flux level may be 50% of the first photon flux level, 20% of the first photon flux level, 10% of the first photon flux level, or the like.

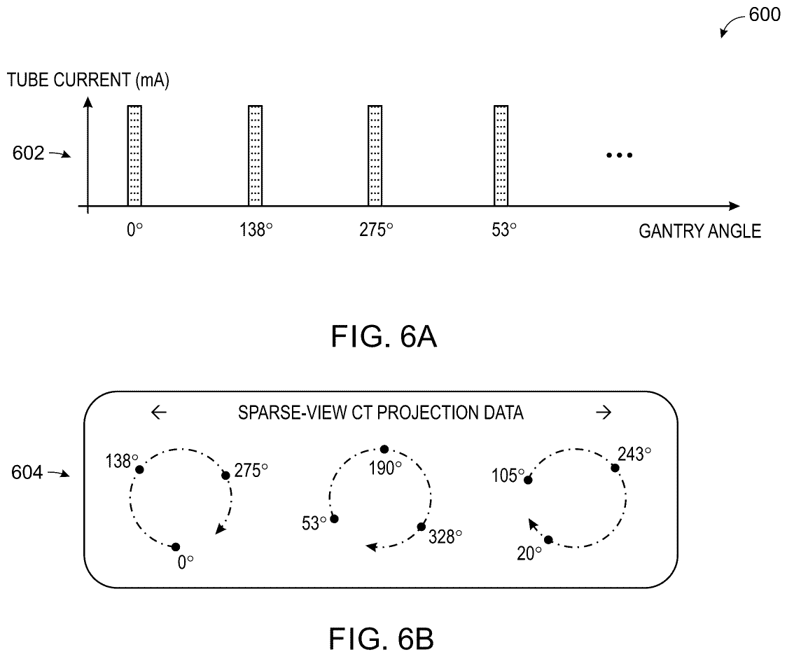

FIGS. 6A and 6B are diagrams 600 of example tube currents of an x-ray source 206 of a CT system 110 for particular gantry angles. As shown in FIGS. 6A and 6B, the angular sequence may be 0°, 138°, 275°, etc. In this case, as shown by reference number 602, the CT system 110 may modulate the X-ray radiation beam by adjusting a tube current of an X-ray tube of the X-ray source 206. Further, the CT system 110 may modulate the X-ray radiation beam such that the X-ray radiation beam is only generated for gantry angles corresponding to the angular sequence. In this way, as shown by reference number 604, the CT system 110 may acquire sparse-view CT projection data for the gantry angles of 0°, 138°, and 275°for a first rotation, may acquire sparse-view CT projection data for the gantry angles of 53°, 190°, and 328°for a second rotation, may acquire sparse-view CT projection data for the gantry angles of 20°, 105°, and 243°for a third rotation, etc.

As further shown in FIG. 5, the process 500 may include generating a CT image of the region of interest and the interventional device 140 using the sparse-view CT projection data and a reconstruction technique (operation 520). For example, the CT system 110 may generate a CT image of the region of interest and the interventional device 140 using the sparse-view CT projection data and a reconstruction technique. The CT image may be a 2D image, a 3D image, or the like.

According to an embodiment, the reconstruction technique may include an AI technique, an iterative reconstruction technique, a weighted iterative reconstruction technique, a reconstruction of difference technique, a stacked back projection technique, a missing spatial-frequency correction technique, a motion correction technique, a registration technique, or the like. Additionally, or alternatively, the reconstruction technique may include a combination of one or more of the foregoing techniques.

According to an embodiment, the CT system 110 may acquire a full-view CT image of the region of interest of the subject and the interventional device 140 for utilization during the generation of the CT image. The full-view CT image may be a CT image that is generated using full-view CT projection data. According to an embodiment, the CT system 110 may acquire the full-view CT projection data during a single rotation. According to an embodiment, the CT system 110 may be configured to acquire the full-view CT projection data based on a predetermined timeframe. For example, the CT system 110 may be configured to acquire the full-view CT projection data every five minutes, every minute, or the like. Additionally, or alternatively, the CT system 110 may be configured to acquire the full-view CT projection data based on an event. For example, the CT system 110 may be configured to acquire the full-view CT projection data based on an initialization of the CT-guided interventional procedure, based on the introduction of the interventional device 140 in the region of interest, based on motion of the interventional device 140 exceeding a threshold, based on a threshold number of rotations being performed, based on a threshold number of sets of sparse-view projection data being acquired, based on an angular sequence being completed, or the like. Additionally, or alternatively, the CT system 110 may be configured to acquire the full-view CT projection data based on an input received via the user input device 218. For example, a clinician may interact with the user input device 218 to cause the CT system 110 to acquire the full-view CT projection data. Additionally, or alternatively, the CT system 110 may be configured to acquire the full-view CT projection data during a preoperative procedure. Alternatively, the CT system 110 may acquire the full-view CT projection data based on aggregating sparse-view CT projection data for multiple rotations. For example, the CT system 110 may aggregate sparse-view CT projection data acquired over multiple rotations such that the aggregated sparse-view CT projection data constitutes a same amount, or a substantially similar amount, of CT projection data as a full-view rotation. For example, the CT system 110 may aggregate sparse-view CT projection data that is acquired during previous rotations, subsequent rotations, or from a different time in a same rotation. Alternatively, the CT system 110 might not acquire full-view CT projection data, and may generate the CT image using only the sparse-view CT projection data.

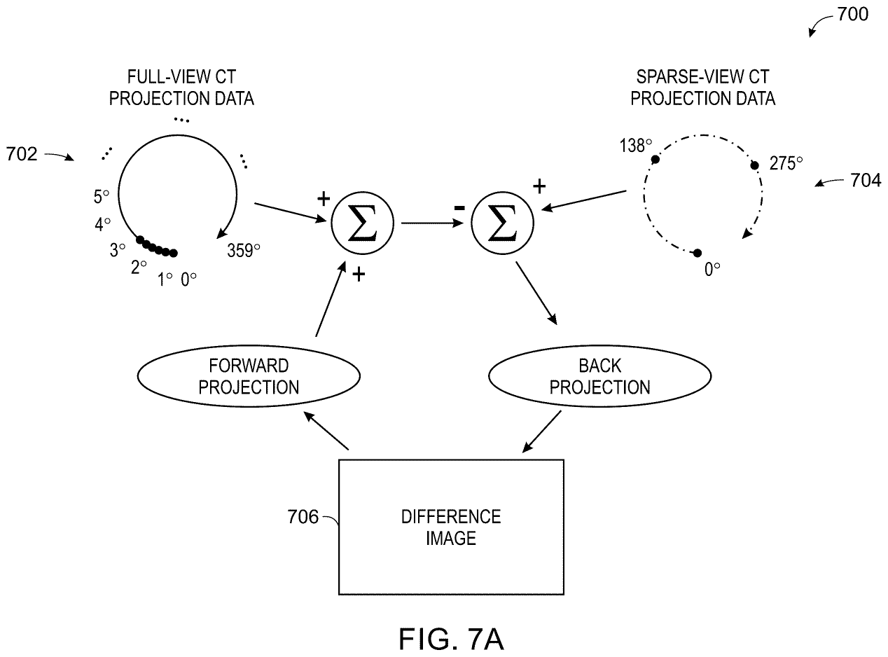

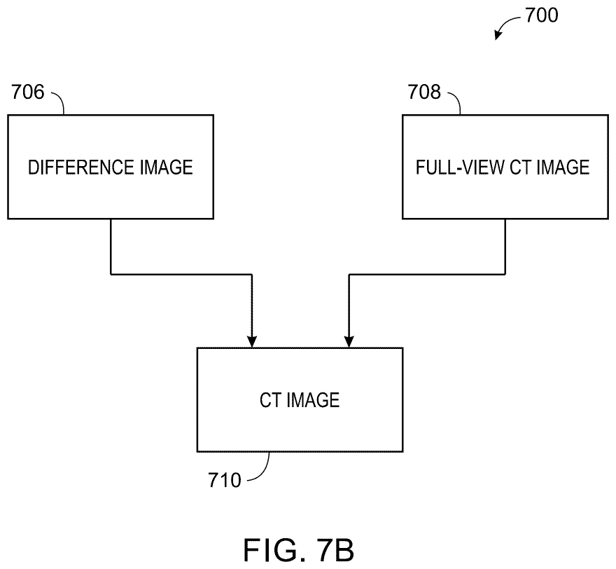

According to an embodiment, the CT system 110 may generate the CT image of the region of interest and the interventional device 140 using the sparse-view CT projection data, a full-view CT image and/or full-view CT projection data, and a reconstruction of difference technique. The difference image may be an image that identifies the differences between the sparse-view CT projection data and the full-view CT image and/or the full-view CT projection data. According to an embodiment, the CT system 110 may generate the difference image by processing the full-view CT image and/or the full-view CT projection data and the sparse-view CT projection data using a reconstruction of difference technique. For example, the CT system 110 may pre-subtract the full-view CT projection data of the full-view CT image from the corresponding sparse-view CT projection data. Alternatively, the CT system 110 may add the full-view CT projection data to corresponding estimated CT projection data. The CT system 110 may generate the CT image based on the full-view CT image and the difference image. FIGS. 7A and 7B are diagrams of an example process 700 for generating a CT image using the sparse-view CT projection data, a full-view CT image, and a reconstruction of difference technique. For example, as shown in FIG. 7A, the CT system 110 may acquire full-view CT projection data 702 and sparse-view CT projection data 704, and may generate a difference image 706 using the full-view CT projection data 702, the sparse-view CT projection data 704, and a reconstruction of difference technique. As shown in FIG. 7B, the CT system 110 may use the difference image 706 and a full-view CT image 708 to generate the CT image 710.

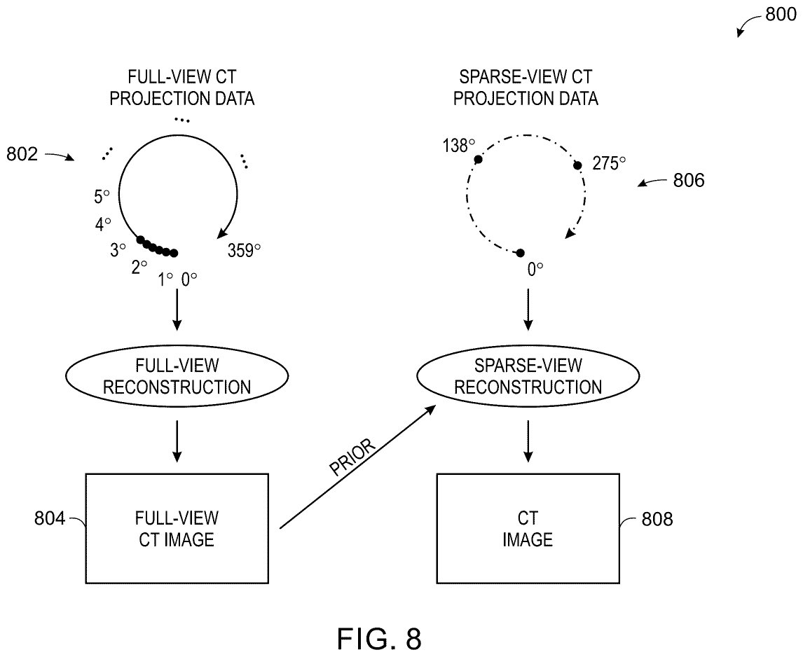

According to an embodiment, the CT system 110 may generate a CT image of the region of interest and the interventional device 140 using the sparse-view CT projection data, a full-view CT image and/or full-view CT projection data, and a reconstruction technique. FIG. 8 is a diagram of an example process 800 for generating a CT image of the region of interest and the interventional device 140 using the sparse-view CT projection data, a full-view CT image and/or full-view CT projection data, and a reconstruction technique. As shown in FIG. 8, the CT system 110 may acquire full-view CT projection data 802, and generate a full-view CT image 804 using a full-view reconstruction technique. Further, as shown in FIG. 8, the CT system 110 may acquire sparse-view CT projection data 806, and generate the CT image 808 using the sparse-view CT projection data 806, the full-view CT image 804, and a sparse-view reconstruction technique, wherein the full-view CT image is used as prior information during the generation of the sparse-view CT image.

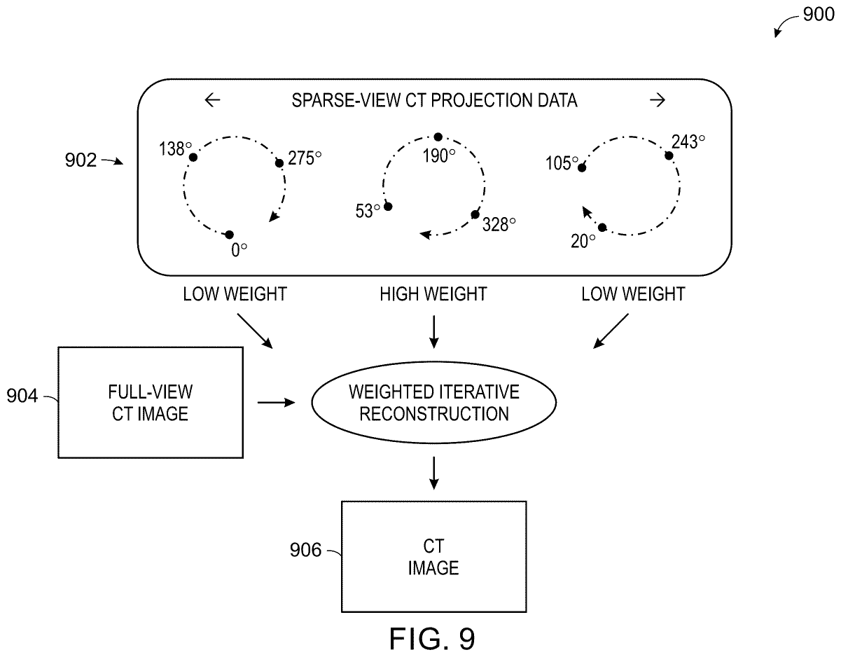

According to an embodiment, the CT system 110 may generate a CT image of the region of interest and the interventional device 140 using the sparse-view CT projection data, a full-view CT image and/or full-view CT projection data, and a weighted iterative reconstruction technique. FIG. 9 is a diagram of an example process 900 for generating a CT image of the region of interest and the interventional device 140 using the sparse-view CT projection data, a full-view CT image, and a weighted iterative reconstruction technique. As shown in FIG. 9, the CT system 110 may acquire sparse-view CT projection data 902. As shown, the CT system 110 may acquire sparse-view CT projection data for a first rotation corresponding to gantry angles of 0°, 138°, and 275°, may acquire sparse-view projection data for a second rotation corresponding to gantry angles of 53°, 190°, and 328°, and may acquire sparse-view CT projection data for a third rotation corresponding to gantry angles of 20°, 105°, and 243°. The CT system 110 may apply various weights to the sparse-view projection data of the respective rotations, and generate the CT image 906 using the sparse-view CT projection data 902, a full-view CT image 904, and a weighted iterative reconstruction technique. The CT system 110 may apply a higher weight for a current-time subset of the sparse-view CT projection data 902. According to another embodiment, the CT system 110 might not utilize the full-view CT image 904 during generation of the CT image 906. According to another embodiment, the CT system 110 may generate a difference image relative to the full-view CT image 904, and use the difference image to generate the CT image 906.

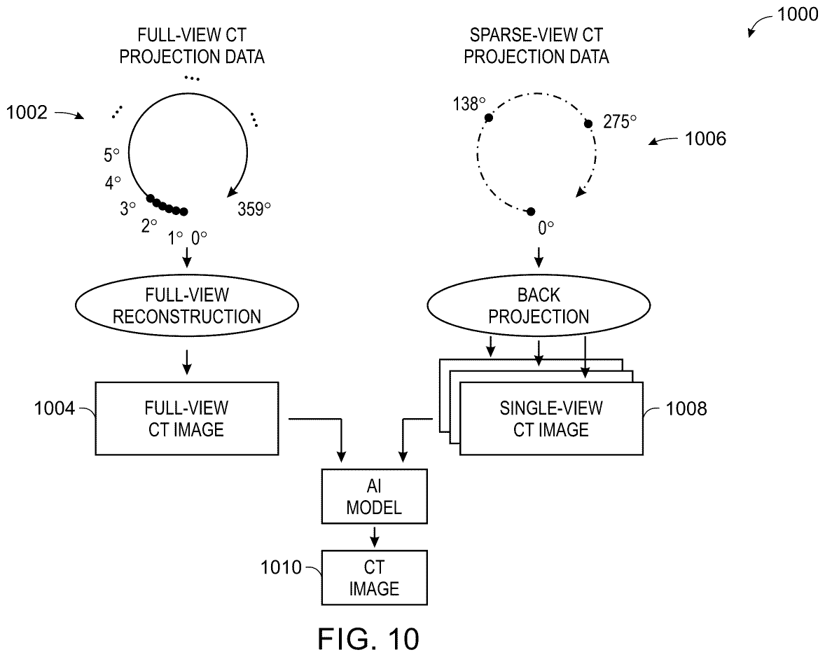

According to an embodiment, the CT system 110 may generate a CT image of the region of interest and the interventional device 140 using the sparse-view CT projection data and a stacked back projection technique. FIG. 10 is a diagram of an example process 1000 for generating a CT image of the region of interest and the interventional device 140 using the sparse-view CT projection data and a stacked back projection technique. As shown in FIG. 10, the CT system 110 may acquire full-view CT projection data 1002, and generate a full-view CT image 1004 using a full-view reconstruction technique. Further, the CT system 110 may acquire sparse-view CT projection data 1006, and generate single-view CT images 1008 using a back projection technique. Further, the CT system 110 may input the full-view CT image 1004 and the single-view CT images 1008 into an AI model that is configured to generate the CT image 1010. According to another embodiment, the CT system 110 might not utilize the full-view CT image 1004 during generation of the CT image 1010. In this case, the AI model may be configured to generate the CT image 1010 using only the single-view CT images 1008. According to another embodiment, the CT system 110 may use an AI model that is configured to reduce post-reconstruction artifacts, such as artifacts from sparse-views, interpolated views, and missing spatial frequencies. Further, according to another embodiment, the CT system 110 may use an AI model that is configured to denoise the CT image 1010. Further, according to yet another embodiment, the AI model may be a hierarchical AI network, generating the CT image in two or more stages that are structured in a tree structure, such that the outputs of several networks in one stage are used as inputs to a single network in a next stage.

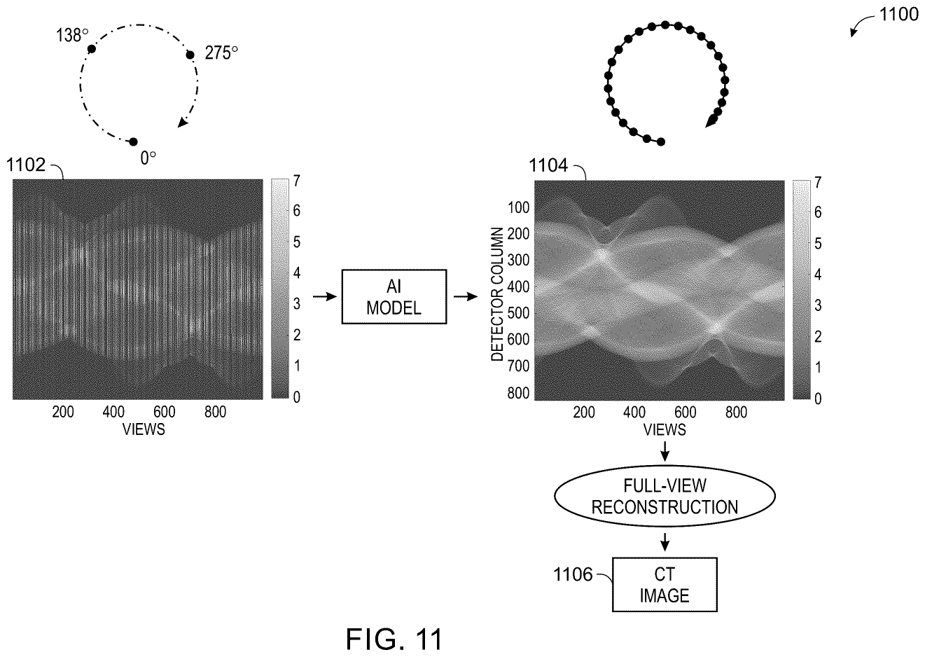

According to an embodiment, the CT system 110 may generate a CT image of the region of interest and the interventional device 140 using the sparse-view CT projection data and an AI technique. FIG. 11 is a diagram of an example process 1100 for generating a CT image of the region of interest and the interventional device 140 using the sparse-view CT projection data and an AI technique. As shown in FIG. 11, the CT system 110 may input a sinogram 1102 corresponding to sparse-view CT projection data into an AI model that is configured to generate a sinogram 1104 corresponding to full-view CT projection data. Further, the CT system 110 may generate a CT image 1106 using the sinogram 1104 and a full-view reconstruction technique. According to another embodiment, the CT system 110 may input a sinogram corresponding to full-view CT projection data into the AI model. According to another embodiment, the CT system 110 may use an AI model that is configured to reduce post-reconstruction artifacts, such as artifacts from sparse-views, interpolated views, and missing spatial frequencies. Further, according to another embodiment, the CT system 110 may use an AI model that is configured to denoise the CT image 1106.

As further shown in FIG. 5, the process 500 may include utilizing the CT image of the region of interest and the interventional device during the CT-guided interventional procedure (operation 530). For example, the CT system 110 may utilize the CT image of the region of interest and the interventional device 140 during the CT-guided interventional procedure. According to an embodiment, the CT system 110 may utilize the CT image of the region of interest and the interventional device 140 by displaying the CT image via the display 216. In this way, a clinician may view the CT image to ascertain the location, orientation, position, or the like, of the interventional device 140 in the region of interest. According to an embodiment, the CT system 110 may utilize the CT image of the region of interest and the interventional device 140 by adjusting an image parameter of the interventional device 140 in the CT image. For example, the image parameter may be a brightness, a color, a hue, a shading, a shadowing, or the like. In this way, a clinician may more readily assess the location, orientation, position, or the like, of the interventional device 140 in the region of interest. According to an embodiment, the CT system 110 may utilize the CT image of the region of interest and the interventional device 140 by providing the CT image to the robotic surgical system 120. In this way, the robotic surgical system 120 may use the CT image to perform the CT-guided interventional procedure.

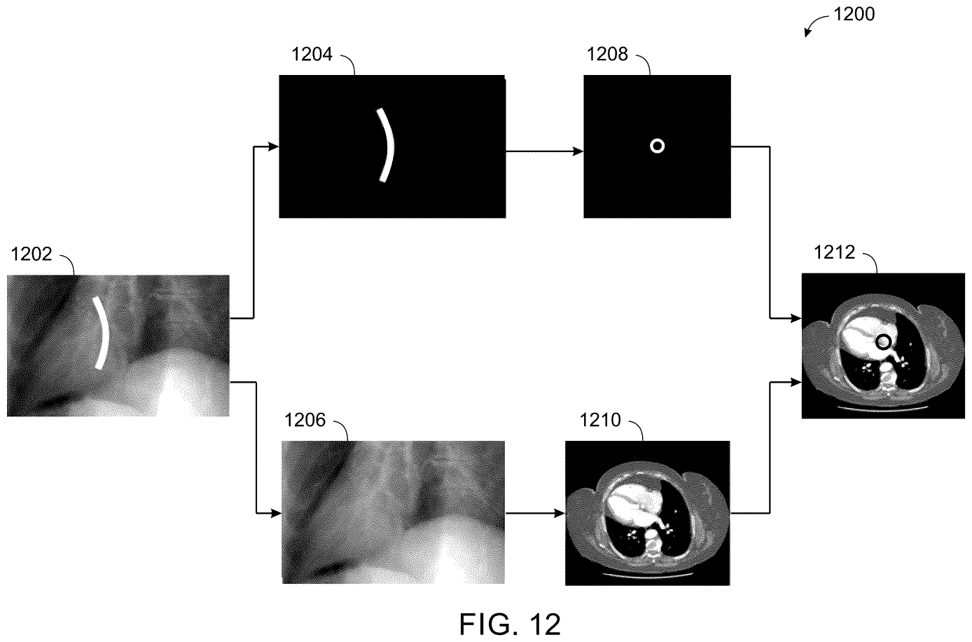

FIG. 12 is a diagram of an example process 1200 for generating a CT image of a region of interest of a subject and an interventional device 140 using sparse-view CT projection data during a CT-guided interventional procedure. As shown in FIG. 12, the CT system 110 may acquire sparse-view CT projection data 1202 of the region of interest of the subject and the interventional device 140. The CT system 110 may separate the sparse-view CT projection data 1202 of the region of interest of the subject and the interventional device 140 into sparse-view CT projection data 1204 corresponding to the interventional device 140, and sparse-view CT projection data 1206 corresponding to anatomical features of the region of interest. The CT system 110 may generate a CT image 1208 of the interventional device 140 using the sparse-view CT projection data 1204 corresponding to the interventional device 140 and a reconstruction technique. For example, the CT system 110 may use a sparse-view reconstruction technique, a segmentation technique, a known-component-reconstruction (KCR) technique, and/or the like. The CT system 110 may generate a CT image 1210 of the anatomical features of the region of interest using the sparse-view CT projection data 1206 corresponding to anatomical features of the region of interest. The sparse-view CT projection data 1206 corresponding to anatomical features of the region of interest may correspond to a broader time range (so corresponding to a less sparse set of CT projection data) than as compared to the sparse-view CT projection data 1204 corresponding to the interventional device 140. The CT system 110 may generate the CT image 1210 of the anatomical features of the region of interest using a reconstruction technique, a motion-compensation technique, a registration technique, and/or the like. The CT system 110 may generate a CT image 1212 by combining the CT image 1208 of the interventional device 140 and the CT image 1210 of the anatomical features of the region of interest. According to another embodiment, the CT system 110 may use the sparse-view CT projection data 1206 corresponding to anatomical features of the region of interest to deformably register a preoperative CT image into a registered CT image for a current time step. The CT system 110 may generate a CT image by combining the CT image 1208 of the interventional device 140 and the registered CT image for the current time step.

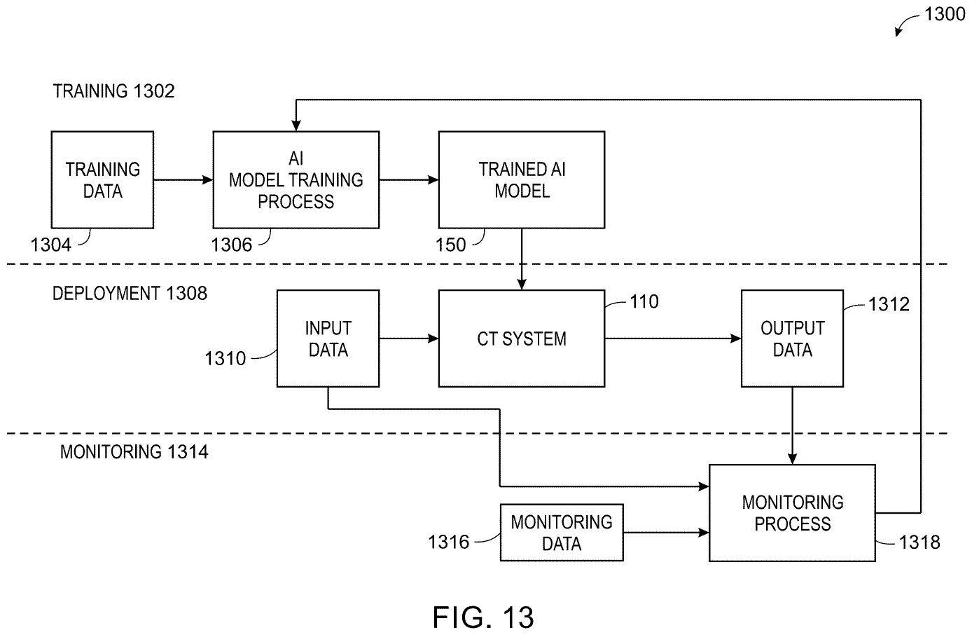

FIG. 13 is a diagram of an example process for training, deploying, and monitoring an AI model 150 for generating a CT image of a region of interest of a subject and an interventional device 140 using sparse-view CT projection data during a CT-guided interventional procedure. The CT system 110 may generate, store, train, and/or use the AI model 150. The AI model 150 may correspond to any one of the AI models described herein. The AI model 150 may be a convolutional neural network (CNN) model, a residual neural network, a random forest model, a decision tree model, an artificial neural network (ANN), a Naïve Bayes model, a decision tree, a recurrent neural network (RNN), a logistic regression model, a support vector machine, a diffusion model, a U-net model, a residual neural network, a generative adversarial network (GAN), or the like.

According to an embodiment, the CT system 110 may include the AI model 150 and/or instructions associated with the AI model 150. For example, the CT system 110 may include instructions for generating the AI model 150, training the AI model 150, using the AI model 150, etc. According to another embodiment, a system or device other than the CT system 110 may be used to generate and/or train the AI model 150. For example, a system or device may include instructions for generating the AI model 150, and/or instructions for training the AI model 150. The system or device may provide a resulting trained AI model 150 to the CT system 110 for use.

As shown in FIG. 13, according to an embodiment, the process 1300 may include a training phase 1302, a deployment phase 1308, and a monitoring phase 1314. In the training phase 1302, at operation 1306, the process 1300 may include receiving and processing training data 1304 to generate a trained AI model 150 for performing one or more operations described herein. The training data 1304 may include sparse-view CT projection data, full-view CT projection data, CT images, difference images, information associated with the interventional device 140, preoperative medical images, sinograms, simulated data, measured data, or the like.

Generally, the AI model 150 may include a set of variables (e.g., nodes, neurons, filters, or the like) that are tuned (e.g., weighted, biased, or the like) to different values via the application of the training data 1304. According to an embodiment, the training process at operation 1306 may employ supervised, unsupervised, semi-supervised, and/or reinforcement learning processes to train the AI model 150. According to an embodiment, a portion of the training data 1304 may be withheld during training and/or used to validate the trained AI model 150.

For supervised learning processes, the training data 1304 may include labels or scores that may facilitate the training process by providing a ground truth. The AI model 150 may have variables set at initialized values (e.g., at random, based on Gaussian noise, based on pre-trained values, or the like). The AI model 150 may provide an output, and the output may be compared with the corresponding label or score (e.g., the ground truth), which may then be back-propagated through the AI model 150 to adjust the values of the variables. This process may be repeated for a plurality of samples at least until a determined loss or error is below a predefined threshold. According to an embodiment, some of the training data 1304 may be withheld and used to further validate or test the trained AI model 150.

For unsupervised learning processes, the training data 1304 may not include pre-assigned labels or scores to aid the learning process. Instead, unsupervised learning processes may include clustering, classification, or the like, to identify naturally occurring patterns in the training data 1304. As an example, the training data 1304 may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data 1304 with pre-assigned labels or scores and training data 1304 without pre-assigned labels or scores may be used to train the AI model 150.

When reinforcement learning is employed, an agent (e.g., an algorithm) may be trained to make a decision regarding the data quality from the training data 1304 through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained AI model 150 may be stored and subsequently applied by the CT system 110 during the deployment phase 1308. For example, during the deployment phase 1308, the trained AI model 150 executed by the CT system 110 may receive input data 1310 for performing one or more operations as described herein.

After being applied by the CT system 110 during the deployment phase 1308, the trained AI model 150 may be monitored during the monitoring phase 1314. The monitoring data 1316 may include data that is output by the AI model 150. During the monitoring phase 1318, the monitoring data 1316 may be analyzed along with the predicted output data 1312 and input data 1310 to determine an accuracy of the trained AI model 150. According to an embodiment, based on the analysis, the process 1300 may return to the training phase 1302, where at operation 1306 values of one or more variables of the model may be adjusted to improve the accuracy of the AI model 150.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. A computed tomography (CT) system comprising:
a memory configured to store instructions; and
one or more processors configured to execute the instructions to:
acquire sparse-view CT projection data of a region of interest of a subject and an interventional device provided within the region of interest of the subject during a CT-guided interventional procedure;
generate a CT image of the region of interest and the interventional device using the sparse-view CT projection data and a reconstruction technique; and
utilize the CT image of the region of interest and the interventional device during the CT-guided interventional procedure.

2. The CT system of claim 1, wherein the one or more processors are further configured to:
control an x-ray source to pulse the x-ray source to acquire the sparse-view CT projection data.

3. The CT system of claim 1, wherein the one or more processors are configured to utilize the CT image by displaying the CT image via a display of the CT system.

4. The CT system of claim 1, wherein the one or more processors are configured to utilize the CT image by providing the CT image to a robotic surgical system that is configured to perform the CT-guided interventional procedure.

5. The CT system of claim 1, wherein the one or more processors are configured to acquire the sparse-view CT projection data using an angular sequence determined by incrementing a binary number in reverse, by gantry angles that are in a golden ratio, or by gantry angles that are in a silver ratio.

6. The CT system of claim 1, wherein the one or more processors are configured to:
generate single-view CT images using the sparse-view CT projection data and a back projection technique; and
generate the CT image using the single-view CT images.

7. The CT system of claim 1, wherein the one or more processors are configured to acquire the sparse-view CT projection data by modulating an X-ray radiation beam such that the X-ray radiation beam is only generated for gantry angles corresponding to an angular sequence.

8. The CT system of claim 1, wherein the one or more processors are configured to acquire the sparse-view CT projection data by modulating an X-ray radiation beam such that an X-ray radiation beam is generated at a first photon flux level for gantry angles corresponding to an angular sequence and is generated at a second photon flux level for gantry angles that do not correspond to the angular sequence.

9. The CT system of claim 1, wherein the one or more processors are configured to generate the CT image using a full-view CT image or full-view CT projection data, the sparse-view CT projection data, and a difference image that identifies differences between the sparse-view CT projection data and the full-view CT image or the full-view CT projection data.

10. The CT system of claim 1, wherein the one or more processors are configured to generate the CT image using a full-view CT image or full-view CT projection data and the sparse-view CT projection data.

11. The CT system of claim 10, wherein the one or more processors are configured to generate the full-view CT image using full-view CT projection data acquired during a single rotation.

12. The CT system of claim 10, wherein the one or more processors are configured to generate the full-view CT image or the full-view CT projection data using the sparse-view CT projection data and additional sparse-view CT projection data acquired during previous rotations, subsequent rotations, or from a different time in a same rotation.

13. The CT system of claim 1, wherein the one or more processors are configured to generate the CT image by applying a weight to the sparse-view projection data.

14. The CT system of claim 1, wherein the one or more processors are configured to generate the CT image by inputting a full-view CT image and a single-view CT image generated using the sparse-view CT projection data into an artificial intelligence model that is configured to generate the CT image.

15. The CT system of claim 1, wherein the one or more processors are configured to generate the CT image by inputting a first sinogram corresponding to the sparse-view CT projection data into an artificial intelligence model that is configured to generate a second sinogram corresponding to full-view CT projection data.

16. The CT system of claim 1, wherein the one or more processors are configured to generate the CT image by separating the sparse-view CT projection data of the region of interest of the subject and the interventional device into first sparse-view CT projection data corresponding to the interventional device and second sparse-view CT projection data corresponding to anatomical features of the region of interest.

17. A method comprising:
acquiring sparse-view computed tomography (CT) projection data of a region of interest of a subject and an interventional device provided within the region of interest of the subject during a CT-guided interventional procedure;
generating a CT image of the region of interest and the interventional device using the sparse-view CT projection data and a reconstruction technique; and
utilizing the CT image of the region of interest and the interventional device during the CT-guided interventional procedure.

18. The method of claim 17, further comprising:
controlling an x-ray source to pulse the x-ray source to acquire the sparse-view CT projection data.

19. A non-transitory computer-readable medium storing instructions that, when executed by one or more processors, cause the one or more processors to:
acquire sparse-view computed tomography (CT) projection data of a region of interest of a subject and an interventional device provided within the region of interest of the subject during a CT-guided interventional procedure;
generate a CT image of the region of interest and the interventional device using the sparse-view CT projection data and a reconstruction technique; and
utilize the CT image of the region of interest and the interventional device during the CT-guided interventional procedure.

20. The non-transitory computer-readable medium of claim 19, wherein the instructions further cause the one or more processors to:
control an x-ray source to pulse the x-ray source to acquire the sparse-view CT projection data.
